(19) United States
(12) Patent Application Publication
(10) Pub. No.: US 2023/0201158 A1
(43) Pub. Date: Jun. 29, 2023

(54) PHARMACEUTICAL COMPOSITION CAPABLE OF INHIBITING REPLICATION OF CORONAVIRUS

(71) Applicant: MAHIDOL UNIVERSITY, Nakhon Pathom (TH)

(72) Inventors: Suparerk BORWORNPINYO, Bangkok (TH); Patoomratana TUCHINDA, Bangkok (TH); Suradej HONGENG, Bangkok (TH); Ampa SUKSATU, Bangkok (TH); Arunee THITITHANYANONT, Bangkok (TH); Suwimon MANOPWISEDJAROEN, Bangkok (TH); Phongthon KANJANASIRIRAT, Bangkok (TH); Bamroong MUNYOO, Bangkok (TH); Napason CHABANG, Bangkok (TH); Noppawan RANGKASENEE, Bangkok (TH); Somchai CHUTIPONGTANATE, Bangkok (TH); Sitthivut CHAROENSUTTHIVARAKUL, Bangkok (TH); Patompon WONGTRAKOONGATE, Bangkok (TH)

(73) Assignee: MAHIDOL UNIVERSITY, Nakhon Pathom (TH)

(21) Appl. No.: 17/999,447

(22) PCT Filed: May 5, 2021
(86) PCT No.: PCT/TH2021/000019

§ 371 Date: Nov. 21, 2022

(30) Foreign Application Priority Data
May 28, 2020 (TH) 2001002981

(51) Int. Cl. A61K 31/353; A61K 31/12; A61K 45/06; A61K 36/906; A61P 31/14

(52) U.S. Cl. CPC A61K 31/353; A61K 31/12; A61K 45/06; A61K 36/906; A61P 31/14

(57) ABSTRACT

The present invention relates to a pharmaceutical composition capable of inhibiting replication of coronavirus in a subject upon administrating the composition in an effective amount to the subject, the composition comprising an extract acquired from a plant part of Boesenbergia sp. having Panduratin A and Pinostrobin in a molar ratio of 1:4 to 1:10. The composition may further comprise or incorporated with or being used along with an antiviral agent to achieve a synergistic effect against coronavirus.

### TECHNICAL FIELD

The present disclosure relates to a pharmaceutical composition, Panduratin A and Pinostrobin, or a plant extract containing the mentioned composition capable of prohibit replication of coronavirus, more specifically severe acute respiratory syndrome coronavirus 2 (SARS-CoV-2) in a human subject upon bringing the composition or the extract into contact with the infected cells or virus. Moreover, the disclosed composition exhibits properties in preventing cells from being infected by the SARS-CoV-2 rendering it an ideal candidate to serve as a prophylactic against SARS-CoV-2 infection as well.

### BACKGROUND

In December 2019, multiple cases of severe pneumonia emerged in Wuhan, Hubei, China [1]. The causative agent was identified to be a novel coronavirus, which was scientifically named severe acute respiratory syndrome coronavirus 2 (SARS-CoV-2). The World Health Organization (WHO) called the disease caused by this virus as coronavirus disease 19 or COVID-19. With the wide and rapid spreading, the virus became pandemic in a short period of time, causing the serious outbreak in 216 countries and territories around the world. As of May 21, 2020, the total confirmed cases of COVID-19 were more than 4,900,000 with more than 320,000 deaths globally [2]. This catastrophic situation highlighted the urgent need of the entire population for the effective and affordable antiviral therapeutics to fight against the dreadful disease.

Basically, SARS-CoV-2 is an enveloped, positive-sense, single-stranded. RNA virus of Coronaviridae family. This virus was categorized as a member of Betacoronavirus genus, alongside severe acute respiratory syndrome coronavirus (SARS-CoV) and Middle East respiratory syndrome coronavirus (MERS-CoV). Usually, most human cases of coronavirus infection are mild or asymptomatic. However, the outbreak of SARS-CoV in 2003 [3, 4], MERS-CoV in 2014 [5], and SARS-CoV-2 at this moment rang the alarm bell of the global public health crisis. Currently, there is no specific drugs for the treatment of COVID-19. All of the drug options are relied on the treatment of the related viruses, such as SARS-CoV, MERS-CoV, influenza virus, Ebola virus, and HIV-1. Accordingly, several FDA-approved drugs with broad therapeutic window serve as the potential candidates for COVID-19 treatment [6, 7]. The most promising repurposed drugs included chloroquine/hydroxychloroquine [8-10], favipiravir [11], lopinavir/ritonavir [12], and remdesivir [13, 14]. However, the degree of efficacy and the severe side effects of these drugs were still under controversy [12, 15, 16]. Apart from FDA-approved drugs, natural product-based medicines are gained much attention. The use of Thai traditional herbs, particularly their phytochemicals, has been reported to exert broad spectrum activities as the anti-cancer, anti-inflammatory, anti-oxidant therapeutics, and antivirals [17-20]. This suggests their potential as the anti-SARS-CoV-2 candidates.

Phytochemicals and plant-derived extracts are ideal places to find a promising drug component against coronavirus [21]. There are several phytochemicals currently under investigation for their applications in treating SARS-CoV-2 as many research groups have recently reported their studies on the potential use of these materials. One of the studies led by Jin Z. et al. [22] demonstrated that the main protease (Mpro) of SARS-CoV-2, a prospective drug target involved in the viral replication and transcription, can be targeted by Shikonin, a common plant-derived naphthoquinone. Further study on the molecular docking showed a reasonable docking pose indicating that Shikonin could bind to the substrate pocket [22]. Khan S A. et. al. employed the computational based methods to identify chymotrypsin-like protease inhibitors (3CL^{Pro}) from FDA-approved antivirals and natural compounds library [23]. Three antiviral drugs (Remdesivir, Saquinavir and Darunavir) and two natural compounds (flavone and coumarin derivatives) were identified as potential inhibitors for 3CL^{Pro} of the coronavirus. Another study on the structure of SARS-CoV-2 3CL^{Pro} has revealed several potential phytochemical flavonoids including myricitrin and licoleafol as inhibitors against this enzyme using the predicted 3D structure [24]. Although these results are encouraging, there are not enough in vitro data to further confirm the benefit and potential of these materials. Further, United States patent publication no. 2011212197 teaches to use aqueous and/or organic extract of at least one Chamomilla plant and/or Achilea plant for the treatment of abnormal proliferation of viral condition in a subject.

In view of these promising findings of the prior researches, it is very likely to discover more potent and ideal therapeutic agents against SARS-CoV-2 infection from natural plants or herbs to increase available arsenal to battle against Covid-19. More importantly, cell-based phenotypic methods combining with high-content imaging technology have dramatically changed the landscape of drug discovery process in recent years. This technique has proven to be valuable and powerful in discovering molecules with desired biological functions in a relevant cell-base setting [25]. Due to the urgent scenario and the prospective potential of phytochemicals as an alternative treatment against novel coronavirus as demonstrated by one or more relevant studies [21, 26], inventors of the present disclosure embarked on a journey in discovering and identifying potential therapeutic agent for SARS-CoV-2 infection from varied plant materials using a high content screening platform.

### SUMMARY

The present disclosure is directed to provide a pharmaceutical composition comprising Panduratin A and Pinostrobin, prepared substantially in a molar ratio of 1:4 to 1:10. The mentioned pharmaceutical composition with the like make-up of Panduratin A and Pinostrobin can be a plant extract of Boesenbergia sp. The disclosed pharmaceutical composition is capable of or can be used for prohibiting progress of viral replication in a subject infected with coronavirus such as SARS, MERS, and/or COVID-19. More particularly, the disclosed composition or the extract prohibits particularly SARS-CoV-2 viral replication in a subject suffering from the coronavirus infection.

Another object of the present disclosure is to disclose a composition, which is preferably plant-based or plant origin, capable of effectively prohibiting replication of coronaviruses in a subject upon administrating an effective amount of the disclosed composition or the extract to the subject.

Further object of the present disclosure is to offer a prophylactic against coronavirus infection by ingesting an effective amount of the disclosed prophylactic within a given period. Particularly, the disclosed prophylactic is plant-based or of plant origin with desirably low toxicity to be incorporated to food or health supplements, which are consumable in a daily fashion to boost the subject immune system against coronavirus coming into contact with the subject.

Further object of the present disclosure associates a plant extract or the use of the plant extract comprising a plant-based therapeutic agent effective against coronavirus. The disclosed plant extract can be used as a supplement, prophylactic, or medicament in the treatment of coronavirus infection. Preferably, the plant extract is acquired via extracting the plant parts of Boesenbergia sp. using one or more polar extraction solvents.

At least one of the preceding objects is met, in whole or in part, by the present disclosure, in which one of the embodiments of the present disclosure relates to a composition comprising Panduratin A and Pinostrobin prepared substantially in a molar ratio of 1:4 to 1:10 for use as a medicament.

In several embodiments, the extract of Boesenbergia sp. and/or the composition of Panduratin A and Pinostrobin is administrated to a subject along or incorporated with an antiviral agent selected from at least one of niclosamide, hydroxychloroquine, ivermectin and favipilavir in order to attain a synergistic therapeutic effect against coronavirus.

In some embodiments, the extract of Boesenbergia sp. or the composition of Panduratin A and Pinostrobin is for use in the treatment of coronavirus infection in a subject. Particularly, the composition prohibits viral replication in the infected subjects pursuant to bringing effective amount of Panduratin A and Pinostrobin into contact with the infected cells of the subject.

Further embodiments of the medicament, the coronavirus is SAR-CoV-1, SAR-CoV-2, or MERS-CoV.

Another aspect of the present disclosure refers to a composition capable of inhibiting replication of coronavirus in a subject upon administrating the composition in an effective or therapeutic amount to the subject. Preferably, the composition comprises a pharmaceutically acceptable substance, Panduratin A and Pinostrobin or a purified polar extract acquired from plant parts of Boesenbergia sp. in an effective amount chemically/physically coupled to the substance. Further, the pharmaceutically acceptable substance is selected from at least one of additive, binder, carrier, diluent, excipient, filler, lubricant, and stabilizer.

More preferably, in several embodiments of the disclosed composition, the Panduratin A and Pinostrobin are in a molar ratio of 1:4 to 1:10 to deliver the desired therapeutic, prophylaxis or immunity boosting effect.

Preferably, the Boesenbergia sp. is selected from Boesenbergia rotunda, Boesenbergia longiflora, and Boesenbergia kingie in some embodiments of the disclosed composition.

In a number of embodiments of the disclosed composition, the plant part is rhizome.

Further embodiments of the disclosed composition, the coronavirus is SAR-CoV-1, SAR-CoV-2, and/or MERS-CoV.

For more embodiments, the composition is a medicament, supplement or prophylactic.

### BRIEF DESCRIPTION OF DRAWINGS

FIG. 1 includes high content imaging analysis of B. rotunda extract, as well as corresponding graph demonstrate percentage of inhibition (red) and percentage of cytotoxic (blue) on the right panel (n=3 biological replicates) with green fluorescent signal being anti-SARS-CoV-2 NP mAb and blue fluorescent being Hoechst stain.

FIG. 2 includes high content image of infected cells treated with hydroxychloroquine and a corresponding graph indicating the percentage of inhibition in red and percentage of cytotoxicity in blue (n=3 biological replicates).

FIG. 3 includes high content image of infected cells treated with Panduratin A and a corresponding graph indicating the percentage of inhibition in red and percentage of cytotoxicity in blue (n=3 biological replicates).

FIG. 4 includes high content image of infected cells treated with Pinostrobin and a corresponding graph indicating the percentage of inhibition in red and percentage of cytotoxicity in blue (n=3 biological replicates).

FIG. 5 includes high content image of infected cells treated with the composition of Panduratin A and Pinostrobin and a corresponding graph indicating the percentage of inhibition in red and percentage of cytotoxicity in blue (n=5 biological replicates).

FIG. 6 are graphs showing results of plaque assay of three candidates: (a) hydroxychloroquine, (b) B. rotunda extract, (c) Panduratin A (n=2 biological replicates)

FIG. 7 includes high content image of B. rotunda extract exhibited anti-SARS-CoV-2 activities in the pre-entry phase and a corresponding graph indicating the percentage of inhibition in red and percentage of cytotoxicity in blue (n=3 biological replicates)

FIG. 8 includes high content image of Panduratin A exhibited anti-SARS-CoV-2 activities in the pre-entry phase and a corresponding graph indicating the percentage of inhibition in red and percentage of cytotoxicity in blue (n=3 biological replicates)

FIG. 9 are graphs showing results of plaque assay of the pre-entry phase: B. rotunda extract and Panduratin A (n=2 biological replicates)

FIG. 10 are graphs showing results on plaque reduction capacity of Panduratin A and Pinostrobin

FIG. 11 includes high content image of infected human alveolar cells treated with Panduratin A and Pinostrobin and a corresponding graph indicating the percentage of inhibition in red and percentage of cytotoxicity in blue (n=3 biological replicates)

### DETAILED DESCRIPTION

The present disclosure may be embodied in other specific forms without departing from its structures, methods, or other essential characteristics as broadly described herein and claimed hereinafter. The described embodiments are to be considered in all respects only as illustrative, and not restrictive. The scope of the disclosure is, therefore, indicated by the appended claims, rather than by the foregoing description. All changes that come within the meaning and range of equivalency of the claims are to be embraced within their scope.

As used herein, the terms “extract” refers to an extract from Boesenbergia sp., and/or fingerroot through bringing out the desired plant-based compounds out of the matrix of plat parts of Boesenbergia sp., and/or fingerroot using one or more polar solvent.

As used herein, the terms “Panduratin A” refers to a chemical agent, compound and/or its derivatives including a purified and/or non-purified form, which may be obtained via chemical synthesis, and/or an extraction from Boesenbergia sp., and/or a fingerroot plant.

As used herein, the terms “Pinostrobin” refers to refers to a chemical agent, compound and/or its derivatives including a purified and/or non-purified form, which may be obtained via chemical synthesis, and/or an extraction from Boesenbergia sp., and/or a fingerroot plant.

As used herein, the term “effective amount” refers to an amount or concentration showing effect of the antiviral activity, particularly coronavirus, either in vitro or in vivo.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the terms “approximately” or “about”, in the context of concentrations of components, conditions, other measurement values, etc., means +/−5% of the stated value, or +/−4% of the stated value, or +/−3% of the stated value, or +/−2% of the stated value, or +/−1% of the stated value, or +/−0.5% of the stated value, or +/−0% of the stated value. As used herein, the phrase “in embodiments” means in some embodiments but not necessarily in all embodiments.

According to one aspect of the present disclosure, a composition comprising Panduratin A and Pinostrobin for inhibiting replication of coronavirus in a subject. More specifically, the Panduratin A and Pinostrobin in the disclosed composition are in a molar ratio of 1:4 to 1:10 is used being the medicament for the treatment of coronavirus in a subject. The subject described in the present disclosure refers to any mammals, particularly human subjects, susceptible towards infection of the coronavirus. It was found by the inventors of the present disclosure that Panduratin A and/or Pinostrobin possesses anti-viral properties effective against coronavirus once the medicament is brought into contact with the cells infected with the coronavirus. Panduratin A and/or Pinostrobin exhibit anti-viral properties against coronavirus by way of prohibiting viral replication within the infected cells of the subject. Despite the mechanisms facilitating the anti-viral effect is yet determined, inventors of the present disclosure offer several actions possibly initiated by the presence of Panduratin A and/or Pinostrobin in the subject infected with coronavirus. Specifically, Panduratin A and/or Pinostrobin may interact with viral protease of the coronavirus as such interaction has been previously reported in some earlier studies about the effect of Panduratin A and/or Pinostrobin towards HIV and Dengue Fever Virus (DENV). Hence, Panduratin A and/or Pinostrobin may function as an inhibitor by competitively binding onto protease of the coronavirus thus interrupting progress of the viral replication in the subject and allow the subject to be recovered from the coronavirus infection. The present disclosure further stipulates that another mechanism likely triggered by Panduratin A and/or Pinostrobin in fighting against coronavirus is by way of antioxidant activities carried out in the infected subject capable of attenuating SARS-CoV-2 infection. The antioxidant activities may further substantiate anti-inflammatory response in the subject to avoid the subject's health condition to deteriorate due to uncontrolled inflammatory reaction induced by SARS-CoV-2 infection. Also, Panduratin A was found to induce autophagy, which plays an important role to restrict viral replication. Still, further research shall determine the possible path taken by the Panduratin A in suppressing SARS-CoV-2 infection via the induction of autophagy.

For a number of embodiments, the coronavirus infection treatable by the disclosed the disclosed composition comprises SAR-CoV-1, SAR-CoV-2, or MERS-CoV.

In several embodiments, the Panduratin A and/or Pinostrobin of the present disclosure can be derived from a plant source or through chemical synthetization. For instance, the Panduratin A and/or Pinostrobin may be extracted from one or more plant parts of the Boesenbergia sp. such as Boesenbergia rotunda Boesenbergia longiflora and/or Boesenbergia kingii. More preferably, Panduratin A and/or Pinostrobin can be acquired using one or more known solvents like water, alcohol, ethyl acetate, acetone, etc. to penetrate matrix of the refined or pulverized plant parts to extract Panduratin A and/or Pinostrobin compounds thereof. The yield and purity of the Panduratin A and/or Pinostrobin extracted can be varied according to the species of Boesenbergia and respective plant parts used in the extraction process. Further purification steps known in the field may be implemented to quantify the yielded Panduratin A and/or Pinostrobin prior to using it as the medicament or manufacturing of the medicament for the treatment of coronavirus infection. For example, optimized high-performance liquid chromatography (HPLC) is employed for Panduratin A and/or Pinostrobin purification in some of the embodiments. Furthermore, the plant part in the present disclosure may comprise rhizomes. Alternatively, it is possible as well to synthesize Panduratin A and/or Pinostrobin chemically via method known in the art.

According to another aspect of the present disclosure, a composition capable of inhibiting replication of coronavirus in a subject upon administrating the composition in an effective amount to the subject. Preferably, the composition further comprises one or more pharmaceutically acceptable substances; and a mixture Panduratin A and Pinostrobin or an extract acquired from plant parts of Boesenbergia sp. Moreover, the one or more pharmaceutically acceptable substance is any one or combination of additive, binder, carrier, diluent, excipient, filler, lubricant, and stabilizer.

It is important to note that the disclosed composition can be a medicament for treating coronavirus infection, a prophylactic to be ingested by the subject for preventing infection of the coronavirus when the subject comes into contact with the virus, or a supplement consumable in a daily manner to boost the subject active immune system to fight against coronavirus when the subject has come into contact with the virus.

Depending on the embodiments, the disclosed composition may comprise additional ingredient or active agents to deliver the therapeutic or immune boosting effect as set out in the present disclosure. For instance, in those embodiments where the disclosed composition adopts the form of a medicament, it may further incorporate with one or more active ingredients other than Panduratin A and/or Pinostrobin to attain optimal therapeutic outcome. The other active ingredients can be an antiviral agent such as niclosamide, hydroxychloroquine, ivermectin and favipilavir, etc. which may preferably, but not necessarily, complementary to anti-viral mechanism taken by the disclosed composition thus enabling various pathways to be triggered in the subject to fight against the coronavirus infection. In embodiments which the disclosed composition is used as supplement, other additional ingredients may comprise Vitamin C, Zinc, and the like to substantiate anti-viral effect of the Panduratin A and/or Pinostrobin or the extract of Boesenbergia sp. To serve as a prophylactic against coronavirus infection, the disclosed composition may comprise like ingredients of the supplement but with different dosage to attain the desired outcome.

As in the setting forth, Panduratin A and/or Pinostrobin or the extract can be obtained by way of reacting the pulverized or refined plant parts of Boesenbergia sp. with a solvent or a polar solvent to dissolve the needed compounds into the polar solvent followed by removing the polar extraction solvent to derive the polar extract containing Panduratin A and/or Pinostrobin. One or more purification steps may be required to quantify and/or purify Panduratin A and/or Pinostrobin from the polar extract. Alternatively, the polar extract can be subjected to quantification as well based upon the concentration or ratio of Panduratin A and/or Pinostrobin in the extract such that the therapeutic or prophylaxis outcome attained by the disclosed composition is in a more controlled and predictable manner. The plant part usable for the extraction is a rhizome.

Despite the inventors of the present disclosure observed that Panduratin A or Pinostrobin in alone also demonstrated great impact on the inhibition of coronavirus replication, the combined utilization of Panduratin A and Pinostrobin, as active ingredients, in many embodiments to prohibit progress of coronavirus infection exhibit synergistic effect, rendering the disclosed composition more potent than other currently commercially available drugs or medication such as ivermectin or hydroxychloroquine in stopping viral replication within the infected subject. Thus, by administrating the disclosed composition to the infected subject as medicament to interrupt the virus replication cycle, the disclosed composition allows the immune system of the subject to have sufficient time to develop active immunity against the coronavirus without exposing the subject to the severe symptoms induced by heavy viral load.

More preferably, in several embodiments, the Panduratin A and Pinostrobin of the disclosed composition are prepared in a molar ratio of 1:4 to 1:10 to deliver the desired therapeutic, prophylaxis or immunity boosting effect.

It is very likely that the disclosed composition competitively inhibits viral protease of the coronavirus rendering the disclosed composition usable to act against relatively broader spectrum of the coronavirus. Particularly, the disclosed composition is effective against the coronavirus comprising SAR-CoV-1, SAR-CoV-2, or MERS-CoV. As described earlier, other active ingredients can be incorporated to the disclosed composition to have better coverage against broad range of coronavirus known in the field to serve as the medicament, prophylactic or supplement.

The following example is intended to further illustrate the disclosure, without any intent for the disclosure to be limited to the specific embodiments described therein.

According to the present disclosure, Vero E6 cells, African green monkey (Cercopithecus aethiops) kidney epithelial cells (ATCC #C1008), were used for the antiviral screening. The cells were grown in Dulbecco's Modified Eagle Medium (DMEM) (Gibco, USA) with 10% fetal bovine serum (FBS) (Gibco, USA). For Vero cells (African green monkey epithelial cells), these cells were cultured in Minimum Essential Medium (MEM) (Gibco, USA) supplemented with 10% FBS and L-glutamine (Gibco, USA). All cultures were grown at 37° C. in 5% CO₂ atmosphere and used in the experiments described in the following examples.

### EXAMPLE 1

Plant materials used for screening were common herbs in Thailand, and most of them were listed in Thai Herbal Pharmacopoeia 2018 (https://bdn.go.th/th/sDetail/10/34/). Boesenbergia rotunda rhizomes were purchased from suppliers in Pathum Thani, Thailand. The plant was identified and compared with depository plant materials of ECDD before starting extraction procedures

Particularly, the air-dried and finely powdered rhizomes of B. rotunda (2.5 kg) were percolated with 95% EtOH (6 L, 4 times×7 days) at room temperature to give a crude EtOH extract (190.5 g) after solvent removal. The obtained EtOH extract was divided into two portions. Each portion was separated by VLC over Si-gel (250 g each, Merck Art. No. 7731), packing on a sintered glass funnel (i.d. 12.5 cm×packing height 4.5 cm), using EtOAc-hexanes and MeOH-EtOAc gradients as eluents, respectively. Fractions (500 mL each) were collected and combined based on their TLC behaviors to give fractions: A1-A5. Fraction A4 (60.1 g, eluted with 25-100% EtOAc-hexanes), after three further consecutive Si-gel CC (Si-gel: Merck, Art. No 7734, 1st CC: 20% EtOAc-hexanes; 2_{nd} CC: 60% CH₂Cl₂-hexanes; 3rd CC: 10% CH₃COCH₃-hexanes) afforded three separated frs. B1-B3. Fr. B3 (5.37 g) was further purified by Sephadex LH-20 CC (Sephadex LH-20: GE Healthcare Bio-Sciences AB, 10% MeOH—CH₂Cl₂), followed by recrystallization from EtOH-CH₂Cl₂ to provide pure Panduratin A (3.18 g).

### EXAMPLE 2

For antiviral activity test against coronavirus, biologically active substances were tested against the coronavirus by employing immunofluorescence cell-based technique that Vero E6 cells were used as a model cell to infect with SARS-CoV-2 in a 96-well plate and incubated at 37° C. for 2 hours. Then the Vero E6 cells were treated with different concentrations of the extract of B. rotunda, Panduratin A, and Pinostrobin at 37° C. for 48 hours period. Thereafter, the culture supernatants were harvested and the cells were fixed and stained with anti-SARS-CoV NP mAb and Alexa Fluor 488-labeled secondary antibody. The cell phenotype has been analyzed by using the High-content imaging system, Operetta, PerkinElmer and the treatment of Hydroxychloroquine was kept as a control group.

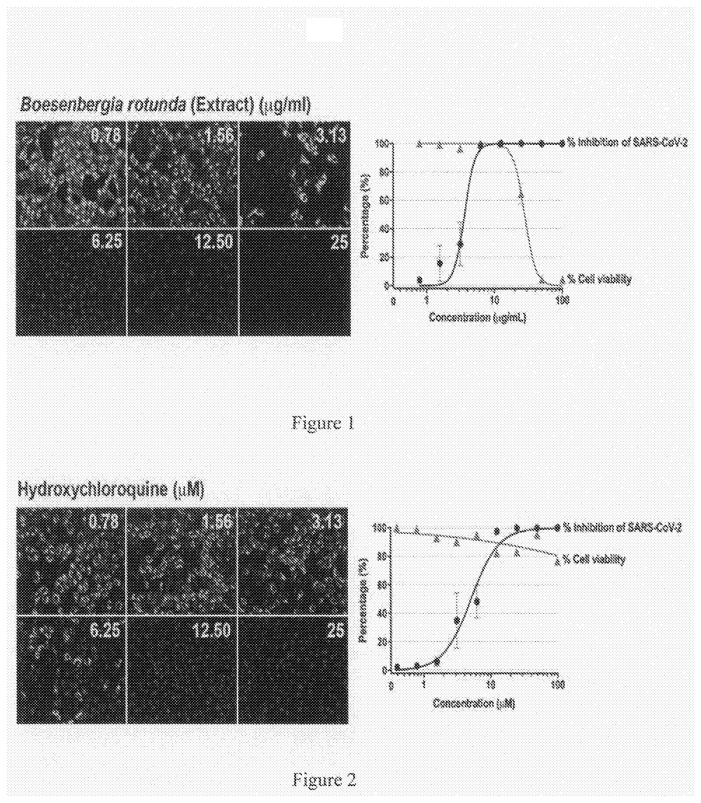

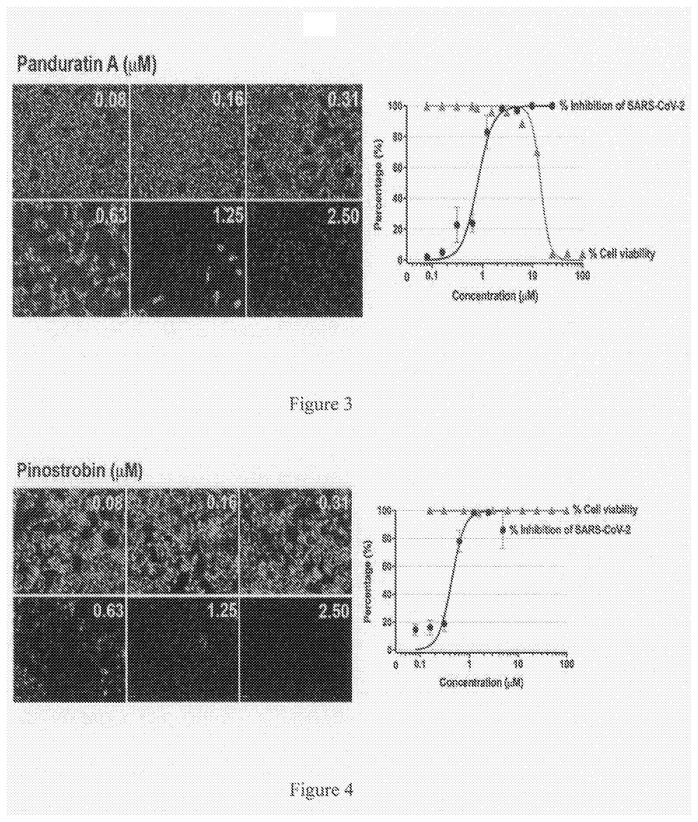

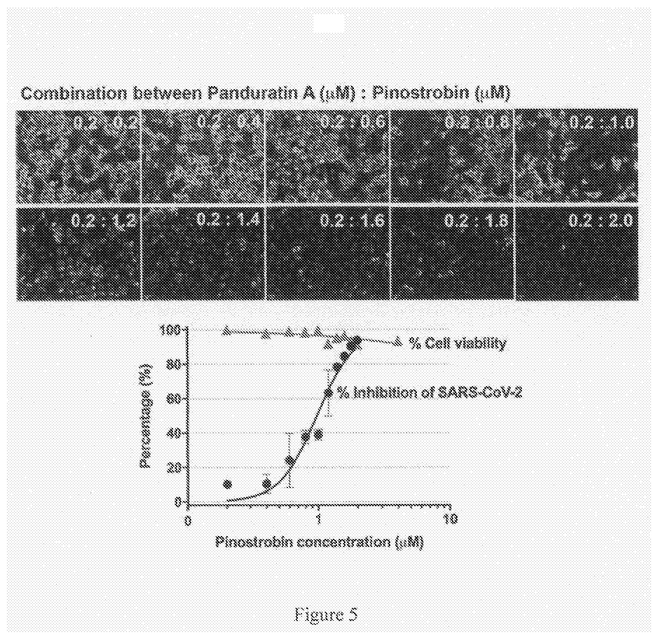

As shown in FIG. 1, the experimental results revealed that extract (at 10 ug/mL) of B. rotunda has inhibitory effects against coronavirus at the active compound concentration of 50% of all virus infected cells (50% Inhibition concentration, IC₅₀) at 3.62 ug/mL. Similarly, Hydroxychloroquine control has 50% inhibition concentration (IC₅₀) of 5.08 uM (1.71 ug/mL) as shown in FIG. 2. Interestingly, the tests of two purified compounds from the extract; Panduratin A (at 10 uM) and Pinostrobin (at 10 uM), revealed better results in coronavirus inhibition than Hydroxychloroquine control by showing 50% inhibition concentration (IC₅₀) at 0.81 uM and 0.44 uM, respectively, as shown in FIG. 3 and FIG. 4. Consecutively, further experiments were performed by combining the two active compounds from the extract at different concentrations of Panduratin A and Pinostrobin in molar ratio as follows: 0.2:0.2, 0.2:0.4, 0.2:0.8, 0.2:1.0, 0.2:1.2, 0.2:1.4, 0.2:1.6, 0.2:1.8, and 0.2:2.0. The extract of B. rotunda comprising Panduratin A and Pinostrobin then exhibited a very potent anti-SARS-CoV-2 activity with IC₅₀ of 0.97 ug/mL, which is higher than that of the Hydroxychloroquine control as shown in FIG. 5.

### EXAMPLE 3

For Cytotoxicity Test by using MTT assay, the Vero E6 cells were cultured in 96-well plates with 10,000 cells per well and kept in an incubator for 24 hours. Then, the cells were incubated with the two active compounds from the B. rotunda extract, Panduratin A and Pinostrobin, in the incubator for 48 hours. When the given time was completed, 100 uL of 0.5 g/mL MTT solvent was added and cells were incubated in the incubator for another 3 hours. MTT solvent was removed and 100 uL DMSO was added. Light absorbance was measured at 570 nm by a microplate reader, and 50% cytotoxicity concentration (CC₅₀) was calculated for each replicate.

The results of cytotoxicity test of the B. rotunda extract against Vero E6 cells revealed 50% cytotoxic concentration (CC₅₀) of 28.06 ug/mL for Hydroxychloroquine control, which is higher than 100 uM as shown in FIG. 1 and FIG. 2. Panduratin A and Pinostrobin have 50% cytotoxic concentrations (CC₅₀) of 14.71 uM and higher than 100 uM, respectively, as shown in FIG. 3 and FIG. 4. Furthermore, the composition of Panduratin A and Pinostrobin has 50% cytotoxic concentration (CC₅₀) higher than 100 uM as shown in FIG. 3, which means that the new combination of these two active compounds of the B. rotunda extract or the fingerroot extract has better inhibitory effects than Hydroxychloroquine control and no cytotoxic effect to the cells (FIG. 5).

### EXAMPLE 4

For the test of Plaque Assay, the Vero E6 cells in 96-well plate were fixed and permeabilized with 50% (v/v) acetone in methanol on ice for 20 min. The cells were washed once with phosphate-buffered saline with 0.5% Tween® detergent (PBST) and blocked in PBST with 2% (w/v) BSA for 1 hour at room temperature. After blocking, the cells were incubated with 1:500 dilution ratio of primary antibody specific for SARS-CoV Nucleoprotein (Rabbit mAb) (Sino Biological Inc. China) for 1 hour at 37° C. Although it was originally against SARS-CoV nucleoprotein (NP), this antibody was able to cross-react with NP protein of SARS-CoV-2 as well. The unbound antibody was removed by washing with PBST for three times. Then, the Goat anti-Rabbit IgG (H+L) Highly Cross-Adsorbed Secondary Antibody, Alexa Fluor 488 (Thermo Fisher Scientific, USA) was used at 1:500 dilution ratio. Nuclei of the cells were stained with Hoechst dye (Thermo Fisher Scientific, USA). The fluorescent signals were detected and analyzed by High-content imaging system (Operetta, PerkinElmer) at 40×. Percentage of the infected cells in each well was automatically obtained from 13 images per well using Harmony software (PerkinElmer).

Furthermore, the viral output of SARS-CoV-2 in the present disclosure was reported as the infectious titers that were determined by plaque assay. In brief, Vero cell monolayer was seeded into 6-well plate 24 hours before the infection. The cells were inoculated with a serial dilution of the virus and incubated for viral adsorption for 1 hour at 37° C. Then, the cells were overlaid with 3 mL/well of overlay medium containing MEM supplemented with 5% FBS and 1% agarose. To allow plaque to develop, the culture was incubated at 37° C. in 5% CO₂ for 3 days. After that, plaque phenotypes were visualized by staining with 0.33% Neutral Red solution (Sigma, USA) for 5 hrs. Plaque numbers were counted and calculated as plaque-forming unit (PFU) per milliliter (mL).

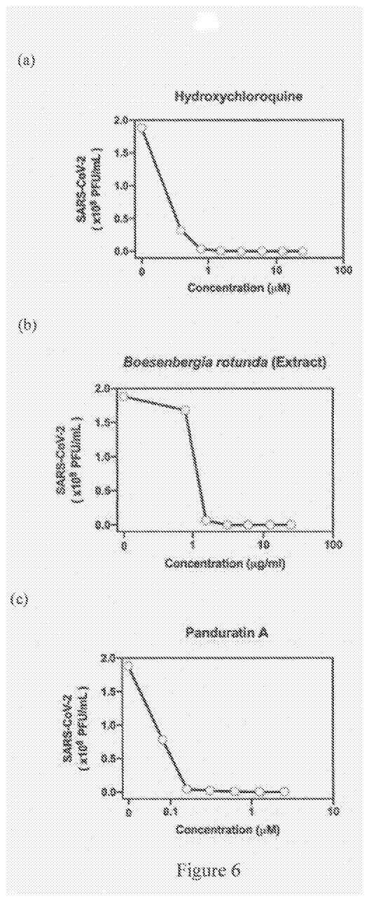

The test results have confirmed that the B. rotunda extract or the fingerroot extracts have inhibitory effects against replication of coronavirus varied to a higher concentration of the Panduratin A. Further, an effective amount or concentration of Panduratin A for treating the viral infected cells is significantly lower than that of Hydroxychloroquine (FIG. 6).

### EXAMPLE 5

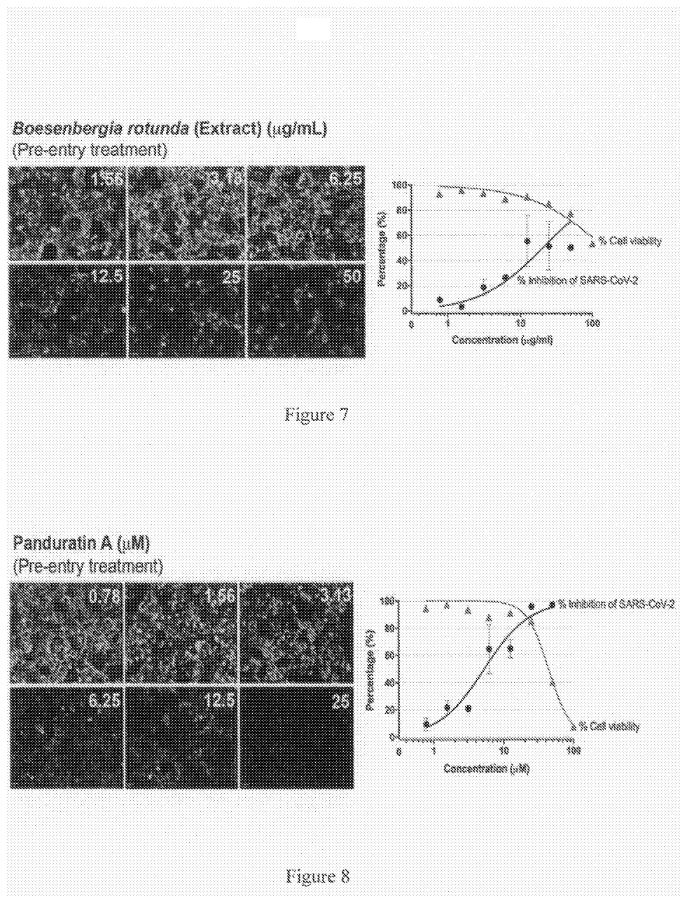

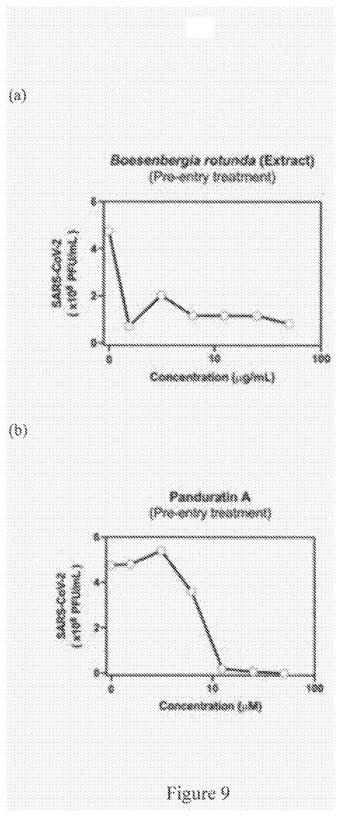

Anti-SARS-CoV-2 effect of B. rotunda extract and Panduratin A at the pre-entry phase. B. rotunda extract and Panduratin A had very potent anti-SARS-CoV-2 activities in the post-infection phase. To extend this impact, it was interesting to know whether B. rotunda extract and Panduratin A also interfere with the viral entry. In this procedure, B. rotunda extract and Panduratin A were pre-incubated with SARS-CoV-2 at 37° C. for 1 h before inoculation into Vero E6 cells. Viral adsorption was allowed for 2 h in the presence of the extract/compound. Then, the cells were washed by fresh medium to remove both the unbound viral particles and the extract/compound. Fresh medium was supplemented and the cells were further cultured for 48 h before harvest. Interestingly, B. rotunda extract and Panduratin A also exhibited anti-SARS-CoV-2 activities in the pre-entry phase. The IC₅₀ of B. rotunda extract and Panduratin A were 20.42 μg/mL (CC₅₀>100 μg/mL) and 5.30 μM (CC₅₀=43.47 μM), respectively (FIG. 7 and FIG. 8). Even though it was less effective than that of post-infection condition, viral output analysis demonstrated approximately five-fold reduction of the infectious virion production following treatment with B. rotunda extract (FIG. 9). Again, Panduratin A absolutely suppressed the infectious virion production at a high dose of 50 μM (FIG. 9).

### EXAMPLE 6

To prove efficacy of B. rotunda extract, Panduratin A and Pinostrobin in the major target cells in human, primary human alveolar cells model was used in plaque assay. The human alveolar cell model was created from primary human alveolar epithelial cells and primary alveolar macrophages cells isolated from lung tissues as previously described by Ruenraroengsak et al. (2005). The primary human alveolar cells were seeded and cultured into each well of a 12-well Transwell™ plate with 1% type I collagen coated plate, in DCCM1 (Cadama, UK), 10% new born calf serum (Invitrogen, UK) and 1% PSG. The primary alveolar macrophages cells were cultured in serum-free RPMI culture medium supplemented with 1% penicillin/streptomycin/I-glutamine (PSG) and plated onto Transwell™ plate. Cell cultures were incubated in 5% CO₂ at 37° C. for cells settle down and adhere to the plate within 48 h of seeding. The medium was removed and the cells were was carefully rinsed with serum free DCCM1 and serum-free RPMI culture medium. After 24 h, cells were inoculated with SARS-CoV-2 at 0.1 multiplicity of infection (MOI) to the apical surface for 2 h. Then, the cultures were washed 3-4 times for remove unbound virus. Apical washed were collected for analysis and tittered by plaque assay.

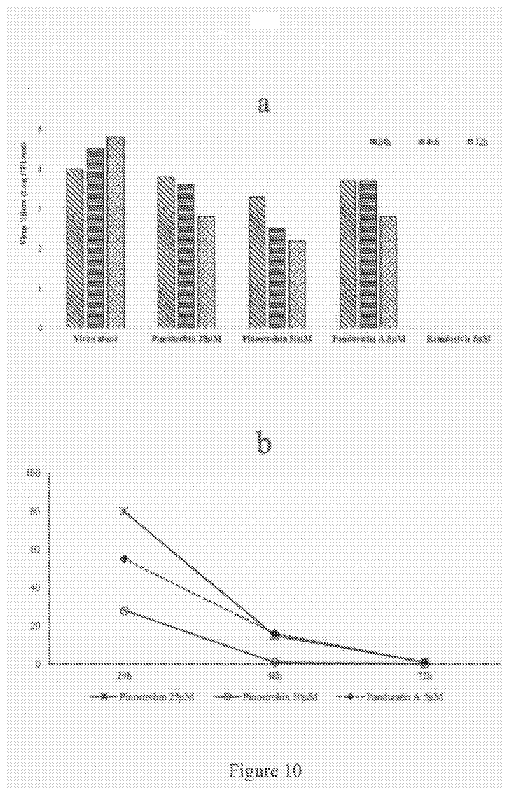

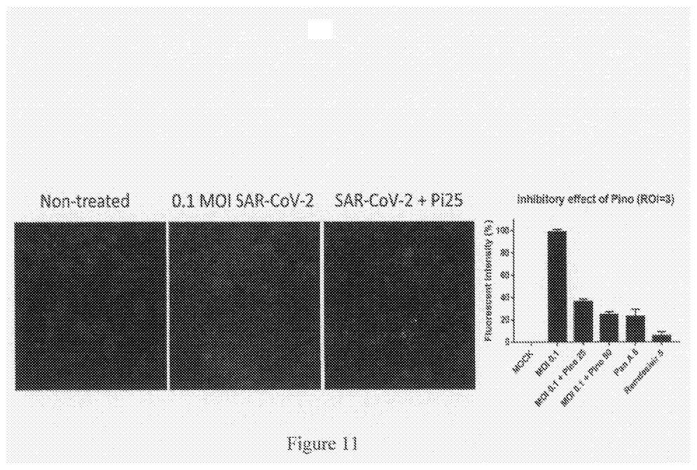

The results showed that Panduratin A and Pinostrobin reduced virus titers in a dose-dependent manner (FIG. 10). At 72 h, virus titer was 4.5 log PFU/mL for vehicle treatment, 2.0 log PFU/mL with 50 μM Pinostrobin. Thus 5 μM Panduratin A and 25 μM Pinostrobin was reduced virus titer in 3 log PFU/mL. Furthermore, analysis of infectious virus by immunofluorescence staining showed that infected cells were reduced 60-70% after treated with 25-50 μM Pinostrobin and/or 5 μM Panduratin A (FIG. 11).

With reference to the above description and examples, the present disclosure provides a novel composition of Panduratin A and Pinostrobin or the extract acquired from the plant part of Boesenbergia sp. for use in inhibiting replication of corona virus, particularly SAR-CoV-2, in a subject. The disclosed composition may not entirely cure the subject from the coronavirus infection, in some circumstances, but administering it to the subject at a predetermined dosage shall substantially reduce the viral load allowing the subject to have more time for developing active immunity against the virus free from suffering from the symptoms triggered by heavy viral load in the body of the subject.

### REFERENCES

1. Huang C, et al. Chronological Changes of Viral Shedding in Adult Inpatients with COVID-19 in Wuhan, China. Clin. Infect. Dis. http://doi:10.1093/cid/ciaa631 (2020).

2. WHO.Coronavirus disease (COVID-19) outbreak situation. https://www.who.int/emergencies/diseases/novel-coronavirus-2019 (accessed May 7, 2020).

3. Smith R D. Responding to global infectious disease outbreaks: lessons from SARS on the role of risk perception, communication and management. Soc Sci Med. 63, 3113-3123. http://doi:10.1016/j.socscimed.2006.08.004 (2006).

4. Anderson R M, et al. Epidemiology, transmission dynamics and control of SARS: the 2002-2003 epidemic. Philos. Trans. R. Soc. Lond. B. Biol. Sci. 359, 1091-1105. http://doi:10.1098/rstb.2004.1490 (2004).

5. Al-Omari A, Rabaan A A, Salih S, Al-Tawfiq J A, Memish Z A. MERS coronavirus outbreak: Implications for emerging viral infections. Diagn. Microbiol. Infect. Dis. 93, 265-285. http://doi:10.1016/j.diagmicrobio.2018.10.011 (2019).

6. Lu H. Drug treatment options for the 2019-new coronavirus (2019-nCoV). Biosci Trends. 14, 69-71. http://doi:10.5582/bst.2020.01020 (2020).

7. Sanders J M, Monogue M L, Jodlowski T Z, Cutrell J B. Pharmacologic Treatments for Coronavirus Disease 2019 (COVID-19): A Review. JAMA. [Online ahead of print] http://doi:10.1001/jama.2020.6019 (2020).

8. Gao J, Tian Z, Yang X. Breakthrough: Chloroquine phosphate has shown apparent efficacy in treatment of COVID-19 associated pneumonia in clinical studies. Biosci. Trends. 14, 72-73. http://doi:10.5582/bst.2020.01047 (2020).

9. Yao X, et al. In Vitro Antiviral Activity and Projection of Optimized Dosing Design of Hydroxychloroquine for the Treatment of Severe Acute Respiratory Syndrome Coronavirus 2 (SARS-CoV-2). Clin. Infect. Dis. http://doi:10.1093/cid/ciaa237 (2020).

10. Colson P, Rolain J M, Lagier J C, Brouqui P, Raoult D. Chloroquine and hydroxychloroquine as available weapons to fight COVID-19. Int. J Antimicrob. Agents. 55, 105932. http://doi:10.1016/j.ijantimicag.2020.105932 (2020).

11. Chen C, et al. Favipiravir versus Arbidol for COVID-19: a randomized clinical trial. medRxiv. Preprint posted Mar. 27, 2020.

12. Cao B, et al. A Trial of Lopinavir-Ritonavir in Adults Hospitalized with Severe Covid-19. N. Engl. J. Med. 382, 1787-1799. http:/doi:10.1056/NEJMoa2001282 (2020).

13. Al-Tawfiq J A, Al-Homoud A H, Memish Z A. Remdesivir as a possible therapeutic option for the COVID-19. Travel. Med. Infect. Dis. 34, 101615. http://doi:10.1016/j.tmaid.2020.101615 (2020).

14. Wang M, et al. Remdesivir and chloroquine effectively inhibit the recently emerged novel coronavirus (2019-nCoV) in vitro. Cell. Res. 30, 269-271. http://doi:10.1038/s41422-020-0282-0 (2020).

15. Costanzo M, De Giglio M A R, Roviello G N. SARS-CoV-2: Recent Reports on Antiviral

Therapies Based on Lopinavir/Ritonavir, Darunavir/Umifenovir, Hydroxychloroquine, Remdesivir, Favipiravir and Other Drugs for the Treatment of the New Coronavirus. Curr Med Chem. http://doi:10.2174/0929867327666200416131117 (2020).

16. Chen J , Liu D , Liu L , et al. A pilot study of hydroxychloroquine in treatment of patients with common coronavirus disease-19 (COVID-19). J. Zhejiang. Univ. 49, 215-219. http://doi:10.3785/j.issn.1008-9292.2020.03.03 (2020).

17. Chusri S, Singthong P, Kaewmanee T. Antioxidant, anticancer, and cytotoxic effects of Thai traditional herbal preparations consumed as rejuvenators. CyTA J. Food. 13, 40-48. http://doi:10.1080/19476337.2014.909885 (2015).

18. Lumlerdkij N, et al. Understanding cancer and its treatment in Thai traditional medicine: An ethnopharmacological-anthropological investigation. J Ethnopharmacol. 216, 259-273. http://doi:10.1016/j.jep.2018.01.029 (2018).

19. Sangkitporn S, et al. Efficacy and safety of zidovudine and zalcitabine combined with a combination of herbs in the treatment of HIV-infected Thai patients. Southeast Asian J. Trop. Med. Public Health. 36, 704-708 (2005).

20. Chotchoungchatchai S, Saralamp P, Jenjittikul T, Pornsiripongse S, Prathanturarug S. Medicinal plants used with Thai Traditional Medicine in modem healthcare services: a case study in Kabchoeng Hospital, Surin Province, Thailand. J Ethnopharmacol. 141, 193-205. http://doi:10.1016/j.jep.2012.02.019 (2012).

21. Mani J S, et al. Natural product-derived phytochemicals as potential agents against coronaviruses: A review. Virus Res. 284, 197989. http://doi:10.1016/j.virusres.2020.197989 (2020).

22. Jin Z, et al. Structure of Mpro from SARS-CoV-2 and discovery of its inhibitors. Nature. http://doi:10.1038/s41586-020-2223-y (2020).

23. Khan S A, Zia K, Ashraf S, Uddin R, Ul-Haq Z. Identification of chymotrypsin-like protease inhibitors of SARS-CoV-2 via integrated computational approach. J. Biomol. Struct. Dyn. 1-10. http://doi:10.1080/07391102.2020.1751298 (2020).

24. Ul Qamar M T, Alqahtani S M, Alamri M A, Chen L L. Structural basis of SARS-CoV-2 3CLpro and anti-COVID-19 drug discovery from medicinal plants. J. Pharm. Anal. http://doi:10.1016/j.jpha.2020.03.009 (2020).

25. Singh S, Carpenter A E, Genovesio A. Increasing the Content of High-Content Screening: An Overview. J. Biomol. Screen. 19, 640-650. http://doi:10.1177/1087057114528537 (2014).

26. Islam M T, et al. Natural products and their derivatives against coronavirus: A review of the non-clinical and pre-clinical data. Phytother. Res. http://doi:10.1002/ptr.6700 (2020).

What is claimed is:

1. A composition capable of inhibiting replication of coronavirus in a subject upon administrating the composition in an effective amount to the subject comprising Panduratin A and Pinostrobin in a molar ratio of 1:4 to 1:10.

2. The composition of claim 1 further comprising a pharmaceutically acceptable substance selected from at least one of additive, binder, carrier, diluent, excipient, filler, lubricant, and stabilizer, wherein the extract is chemically and/or physically coupled to the carrier.

3. The composition of claim 1 further comprising an antiviral agent selected from at least one of niclosamide, hydroxychloroquine, ivermectin and favipilavir.

4. The composition of claim 1, wherein the coronavirus is selected from SAR-CoV-1, SAR-CoV-2, and MERS-CoV.

5. The composition of claim 1 is an extract acquired from a plant part of Boesenbergia sp.

6. The composition of claim 5, wherein the plant part is a rhizome.

7. The composition of claim 5, wherein the Boesenbergia sp. is selected from Boesenbergia rotunda, Boesenbergia longiflora and Boesenbergia kingii.

8. The composition of claim 1 is a medicament, supplement or prophylactic.

9. An extract acquired from a plant part of Boesenbergia sp. comprising Panduratin A and Pinostrobin in a molar ratio of 1:4 to 1:10 for use in the treatment of coronavirus infection in a subject by way of inhibiting replication of the coronavirus.

10. The extract of claim 9 further comprising an antiviral agent selected from at least one of niclosamide, hydroxychloroquine, ivermectin and favipilavir.
